(19) United States
(12) Patent Application Publication
(10) Pub. No.: US 2010/0256747 A1
(43) Pub. Date: Oct. 7, 2010

(54) IMPLANT OF A BIOCORRODIBLE METALLIC MATERIAL AND ASSOCIATED PRODUCTION METHOD

(76) Inventors: Timo Hausbeck, Erlangen (DE); Alexander Rzany, Nuernberg (DE)

Correspondence Address: GREER, BURNS & CRAIN, 300 S WACKER DR, 25TH FLOOR, CHICAGO, IL, 60606, US

(21) Appl. No.: 12/717,617

(22) Filed: Mar. 4, 2010

(30) Foreign Application Priority Data
Apr. 2, 2009 (DE) 10 2009 002 153.1

(51) Int. Cl. A61F 2/82; A61F 2/02; B23P 17/00

(52) U.S. Cl. 623/1.46; 623/23.75; 29/527.2

(57) ABSTRACT

The invention relates to an implant of a biocorrodible metallic material with a passivating coating of a nanoparticle-containing silane coating and an associated method for producing the implant. To this end, the method comprises the steps:
(i) Providing a blank for the implant comprising the biocorrodible metallic material, and
(ii) Coating the blank with a homogenous colloidal solution of
   A) one or more bis-silanes of the formula (1):

(RO)₃—Si—X—Si(OR)₃ (1)
wherein R stands individually selected for C₁-C₁₀ alkyl or C₂-C₁₀ acyl; and
X is a substituted or unsubstituted C₁-C₂₀ alkanediyl group or a substituted or unsubstituted C₁-C₂₀ heteroalkanediyl group with 1 to 5 heteroatoms selected from the group O, N and S; and
B) Silicon dioxide nanoparticles with an average particle diameter in the range of 10 nm to 1 μm
   in water, ethanol or a mixture of the same, wherein the solution contains 1 to 20% by volume bis-silane and the concentration of the silicon dioxide nanoparticles in the solution is in the range of 1 to 100 ppm.

### FIELD OF THE INVENTION

The invention relates to an implant of a biocorrodible metallic material with a passivating coating of a silicon compound and an associated method for producing the implant.

### BACKGROUND OF THE INVENTION

Medical implants for a wide variety of purposes are known in the prior art. Frequently, the implant is required to remain only temporarily in the body to fulfill the medical purpose. Implants of permanent materials, i.e., materials that are not degraded in the body, have to be removed again in many applications because rejection reactions of the body may occur in the medium and long term, even where there is high biocompatibility.

One approach for avoiding leaving the implant in the body permanently or further surgical intervention to remove the same now lies in forming the implant entirely or in parts of a biocorrodible material. Biocorrosion means microbial processes or processes caused simply by the presence of bodily media, which result in a gradual degradation of the structure comprising the material. At a certain point in time, the implant, or at least the part of the implant made of the biocorrodible material, loses its mechanical integrity. The degradation products are largely absorbed by the body, small quantities of residue being tolerable.

Biocorrodible materials have been developed, inter alia, on the basis of polymers of a synthetic nature or natural origin. The material properties, but also in part the biocompatibility of the degradation products of the polymers, limit the use significantly, however. Thus, for example, orthopedic implants must frequently withstand high mechanical stresses; and vascular implants, e.g. stents, must meet very special requirements for modulus of elasticity, brittleness, and deformability depending on design.

A promising approach for solving the problem lies in the use of biocorrodible metal alloys. Thus, it is suggested in DE 197 31 021 A1 that medical implants be molded from a metallic material, the main component of which is an element from the group alkali metals, alkaline earth metals, iron, zinc and aluminum. Alloys based on magnesium, iron and zinc are described as especially suitable. Secondary components of the alloys may be manganese, cobalt, nickel, chromium, copper, cadmium, lead, tin, thorium, zirconium, silver, gold, palladium, platinum, silicon, calcium, lithium, aluminum, zinc and iron. Furthermore, the use of a biocorrodible magnesium alloy having a proportion of magnesium >90%, yttrium 3.7-5.5%, rare earth metals 1.5-4.4%, and the remainder <1% is known from DE 102 53 634 A1, which is suitable, in particular, for producing an endoprosthesis, e.g., in the form of a self-expanding or balloon-expandable stent. Notwithstanding the progress achieved in the field of biocorrodible metal alloys, the alloys known up to this point are capable only of restricted use because of their corrosion behavior. The relatively rapid biocorrosion of magnesium alloys limits their use.

The fundamentals of magnesium corrosion as well as a large number of technical methods for improving corrosion behavior (in the sense of reinforcing the corrosion protection) are known from the prior the art. It is known, for example, that the addition of yttrium and/or further rare earth metals to a magnesium alloy provides a slightly increased corrosion resistance in seawater.

One approach provides producing a corrosion-protecting layer on the molded body comprising magnesium or a magnesium alloy. Known methods for producing a corrosion-protecting layer have been developed and optimized from the aspect of a technical use of the molded body, but not a medical-technical use in biocorrodible implants in a physiological environment. These known methods comprise the application of polymers or inorganic cover layers, the production of an enamel, the chemical conversion of the surface, hot gas oxidation, anodization, plasma spraying, laser beam remelting, PVD methods and ion implantation.

Conventional technical areas of use for molded bodies made of magnesium alloys outside medical technology normally require extensive suppression of corrosive processes. Accordingly, the goal of most technical methods is complete inhibition of corrosive processes. In contrast, the goal for improving the corrosion behavior of biocorrodible magnesium alloys is not the complete suppression, but only the inhibition of corrosive processes. For this reason alone, most known methods are not suitable for producing a corrosion protection layer. Furthermore, toxicological aspects must also be taken into consideration for a use in medical technology. Moreover, corrosive processes are strongly dependent on the medium in which they occur, and, therefore, it is not unrestrictedly possible to apply the findings on corrosion protection obtained under conventional environmental conditions in the technical field to the processes in a physiological environment. Finally, in a plurality of medical implants, the mechanisms on which the corrosion is based may also deviate from typical technical applications of the material. Thus, for example, stents, surgical suture material, or clips are mechanically deformed in use, so that the partial process of tension cracking corrosion may have great significance in the degradation of these molded bodies.

DE 101 63 106 A1 provides changing the magnesium material in its corrosivity by modification with halogenides. The magnesium material is to be used for producing medical implants. The halogenide is preferably a fluoride. The material is modified by alloying halogen compounds in salt form. The composition of the magnesium alloy is accordingly changed by adding the halogenides to reduce the corrosion rate. Accordingly, the entire molded body comprising a modified alloy of this type will have an altered corrosion behavior. However, further material properties, which are significant in processing or also affect the mechanical properties of the molded body produced from the material, may be influenced by the alloying.

Furthermore, coatings for implants made of non-biocorrodible, i.e., permanent materials, are known, which are based on organosilicon compounds. Thus, for example, DE 699 12 951 T2 describes an intermediate layer made of a functionalized silicone polymer, such as siloxanes or polysilanes. US 2004/0236399 A1 discloses a stent having a silane layer, which is covered by a further layer.

DE 10 2006 038 231 A1 describes an implant of a biocorrodible metal material with a coating of an organosilicon compound. The coating can be produced, i.a., using a bis-silane.

### DESCRIPTION OF THE INVENTION

The object of the present invention is to solve or at least to alleviate one or more of the problems described above. In particular, a coating is to be provided for an implant of a biocorrodible material, which coating causes a temporary inhibition, but not complete suppression of the corrosion of the material in a physiological environment.

This object is attained according to a first aspect of the invention through the method according to the invention for producing an implant from a biocorrodible metallic material with a nanoparticle-containing silane coating. To this end, the method comprises three steps:

(i) Providing a blank for the implant comprising the biocorrodible metallic material; and

(ii) Coating the blank with a homogenous colloidal solution of
A) one or more bis-silanes having the formula (1):

(RO)₃—Si—X—Si(OR)₃ (1)
R standing individually selected for C₁-C₁₀ alkyl or C₂-C₁₀ acyl; and
X is a substituted or unsubstituted C₁-C₂₀-alkanediyl group or a substituted or unsubstituted C₁-C₂₀ heteroalkanediyl group with 1 to 5 heteroatoms selected from the group O, N and S; and
B) Silicon dioxide nanoparticles with an average particle diameter in the range of 10 nm to 1 μm
   in water, ethanol or a mixture thereof, wherein the solution contains 1 to 20% by volume bis-silane and the concentration of the silicon dioxide nanoparticles in the solution is in the range of 1 to 100 ppm.

It has now been shown that through the combination of one or more bis-silanes of the above-referenced formula with SiO₂ nanoparticles a corrosion-inhibiting coating can be produced on the base body of the biocorrodible metallic material that is advantageous for the intended purposes. Presumably, through the presence of SiO₂ nanoparticles in the coating hydroxide ions are bonded to the substrate surface, forming a passivating silicate. Cathodic reactions are thereby inhibited in the corrosion of the materials magnesium, iron, zinc or tungsten, so that the corrosion is temporarily inhibited.

Preferably, R is respectively selected individually from the group comprising methyl, ethyl, propyl and i-propyl. In particular, the solution contains bis-[3-(triethoxysilyl)-propyl]tetrasulfide and/or bis-[3-(trimethoxysilylpropyl)]amine. If bis-[3-(trimethoxysilylpropyl)]amine is used, the solution preferably contains vinyltriacetoxysilane, for example, as 4:1 mixture of bis[3-(trimethoxysilylpropyl)]amine and vinyltriacetoxysilane, in order to adjust the pH value of the coating solution. Nanoparticle-containing silane films as corrosion protection for aluminum alloys were already presented by V. Palanivel et al. at the conference The Workshop on Nanoscale Approaches for Multifunctional Coatings, Keystone, Colo., USA, Aug. 12-16, 2002. Preferably X furthermore stands for a C₂-C₁₀ alkanediyl group (also archaically referred to as alkylene group) or a substituted or unsubstituted C₂-C₁₀ heteroalkanediyl group with 1 to 3 heteroatoms selected from the group O, N and S.

According to a further preferred embodiment of the method, which can also be carried out in particular in combination with the aforementioned variants, the step (ii) of the coating is repeated n-fold, wherein n=2 to 10 and the homogenous colloidal solution used in each repetition step can be freely selected in terms of its composition. In other words, the coating is produced by the sequential application of identical or different homogenous colloidal solutions. For example, first a bis[3-(triethoxysilyl)-propyl]tetrasulfide solution can be used, then a solution that contains bis-[3-(trimethoxysilylpropyl)]amine and vinyltriacetoxysilane and finally a bis[3-(trimethoxysilylpropyl)]amine solution.

Preferably, the biocorrodible metallic material is a biocorrodible alloy selected from the group of elements magnesium, iron, zinc and tungsten; in particular the material is a biocorrodible magnesium alloy. In this case an alloy is a metallic structure, the main component of which is magnesium, iron, zinc or tungsten. The main component is the alloy component, the weight proportion of which in the alloy is highest. A proportion of the main component is preferably more than 50% by weight, in particular more than 70% by weight.

The alloys of the elements magnesium, iron, zinc or tungsten are to be selected in their composition such that they are biocorrodible. For the purpose of the present invention, alloys are referred to as biocorrodible in which in a physiological environment a degradation occurs, which ultimately leads to the entire implant or the part of the implant made from the material losing its mechanical integrity. Artificial plasma, as stipulated for biocorrosion tests under EN ISO 10993-15:2000 (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l), is used as test medium for testing the corrosion behavior of an alloy coming into consideration. For this purpose, a sample of the alloy to be tested is stored in a closed sample container with a defined quantity of the test medium at 37° C. At time intervals—coordinated with the anticipated corrosion behavior—of a few hours to several months, the samples are removed and tested for traces of corrosion in the known manner. The artificial plasma according to EN ISO 10993-15:2000 corresponds to a hematoid medium and thus represents a possibility of simulating a physiological environment in terms of the invention in a reproducible manner.

In this case the term corrosion relates to the reaction of a metallic material with its environment, a measurable change to the material being caused, which—when the material is used in a component—leads to an impairment of the function of the component. A corrosion system in this case comprises the corroding metallic material as well as a liquid corrosion medium, which in its composition reproduces the conditions in physiological environment or is a physiological medium, in particular blood. In terms of the material, corrosion is influenced by factors, such as the composition and pretreatment of the alloy, microscopic and submicroscopic inhomogeneities, boundary zone properties, temperature state and stress state and, in particular, the composition of a layer covering the surface. In terms of the medium, the corrosion process is influenced by conductivity, temperature, temperature gradients, acidity, volume-surface ratio, concentration difference as well as flow rate.

Redox reactions take place at the phase boundary between material and medium. For a protective or inhibiting effect, existing protective layers and/or the products of the redox reactions must form a sufficiently tight structure against the corrosion medium, must have an increased thermodynamic stability based on the environment and must be hardly soluble or insoluble in the corrosion medium. In the phase boundary, to be more precise, in a double layer forming this region, adsorption and desorption processes take place. The processes in the double layer are marked by the cathodic, anodic and chemical partial processes occurring there. In the case of magnesium alloys, a gradual alkalinization of the double layer can generally be observed. Foreign material deposits, contaminants and corrosion products influence the corrosion process. The processes during corrosion are accordingly highly complex and cannot be predicted at all or can be predicted only to a limited extent particularly in connection with a physiological corrosion medium, i.e., blood or artificial plasma, because there are no comparison data. For this reason alone, the discovery of a corrosion-inhibiting coating, i.e., a coating that is used only for the temporary reduction of the corrosion rate of a metallic material of the above-referenced composition in physiological environment, is a measure outside the routine of one skilled in the art. This applies in particular to stents, which are exposed to locally high plastic deformations at the time of implantation. Conventional approaches with rigid corrosion-inhibiting layers are unsuitable under conditions of this type.

The process of corrosion can be quantified by specifying a corrosion rate. A rapid degradation is associated with a high corrosion rate, and vice versa. A surface modified in terms of the invention will lead to a reduction in the corrosion rate with regard to the degradation of the entire molded article. The corrosion-inhibiting coating according to the invention can be broken down itself over the course of time or can protect the regions of the implant covered thereby only to a steadily decreasing extent. The course of the corrosion rate is therefore not linear for the entire implant. Instead, at the beginning of the occurring corrosive processes, a relatively low corrosion rate results, which increases over time. This behavior is understood to be a temporary reduction of the corrosion rate for the purposes of the invention and distinguishes the corrosion-inhibiting coating. In the case of coronary stents, the mechanical integrity of the structure is to be maintained over a period of three to six months after implantation.

Another aspect of the invention relates to the an implant, which can be produced or is produced according to the above-referenced method. For the purposes of the invention, implants are devices inserted into the body via a surgical method and comprise fasteners for bones, such as screws, plates, or nails, surgical suture material, intestinal clamps, vascular clips, prostheses in the area of the hard and soft tissue, occluders and anchoring elements for electrodes, in particular, of pacemakers or defibrillators. The implant is composed entirely or in part of the biocorrodible material. When the implant is composed only in part of the biocorrodible material, this part must be coated accordingly.

The implant is preferably a stent. Stents of conventional construction have filigree structure made of metallic struts, which is first provided in an unexpanded state for introduction into the body and which is then expanded into an expanded state at the location of application. Special requirements exist for the corrosion-inhibiting layer in stents: the mechanical stress of the material during the expansion of the implant has an impact on the course of the corrosion process, and it can be assumed that the stress-crack corrosion will be greater in the stressed regions. A corrosion inhibiting layer should take this fact into consideration. Furthermore, a hard corrosion-inhibiting layer could chip off during the expansion of the stent and a crack formation in the layer during expansion of the implant is likely to be unavoidable. Finally, the dimensions of the filigree metallic structure must be noted and as far as possible only a thin, yet also uniform corrosion-inhibiting layer should be produced. It has now been shown that the application of the coating according to the invention meets these requirements in full or at least largely.

The functionalities necessary for binding the bis-silanes can be produced by a pretreatment on the implant surface, for example, through a plasma treatment in a high-oxygen or high-nitrogen atmosphere or a brief bath in caustic lye of soda.

Another aspect of the invention relates to an implant of a biocorrodible metallic material with a nanoparticle-containing silane coating, wherein the coating comprises
A) one or more bis-silanes of the formula (1):

(RO)₃—Si—X—Si(OR)₃ (1)

wherein R stands individually selected for C₁-C₁₀ alkyl or C₂-C₁₀ acyl; and
X is a substituted or unsubstituted C₁-C₂₀ alkanediyl group or a substituted or unsubstituted C₁-C₂₀ heteroalkanediyl group with 1 to 5 heteroatoms selected from the group O, N and S; and
B) Silicon dioxide nanoparticles with an average particle diameter in the range of 10 nm to 1 μm.

Preferably R is respectively chosen individually from the group comprising methyl, ethyl, propyl and i-propyl. In particular, the bis-silane is bis-[3-(triethoxysilyl)-propyl]tetrasulfide and/or bis[3-(trimethoxysilylpropyl)]amine.

Preferably X further stands for a C₂-C₁₀ alkanediyl group or a substituted or unsubstituted C₂-C₁₀ heteroalkanediyl group with 1 to 3 heteroatoms selected from the group O, N and S.

According to a preferred embodiment of the implant, the implant has one or more layers, of the same or different composition, comprising one or more bis-silanes of the formula (1) and silicon dioxide nanoparticles, in particular the implant has n coatings, wherein n=2 to 10.

Preferably, the biocorrodible metallic material is a biocorrodible alloy selected from the group of elements magnesium, iron, zinc and tungsten, in particular the material is a biocorrodible magnesium alloy. In this case, an alloy is understood to mean a metallic structure, the main component of which is magnesium, iron, zinc or tungsten. The main component is the alloy component, the weight proportion of which in the alloy is highest. A proportion of the main component is preferably more than 50% by weight, in particular more than 70% by weight.

Preferably the implant is a stent.

The invention is explained in more detail below based on exemplary embodiments.

### EXEMPLARY EMBODIMENT 1

### Coating of a Stent with a bis-sulfur silane

Stents made of the biocorrodible magnesium alloy WE43 (93% by weight magnesium, 4% by weight yttrium (W) and 3% by weight rare earth metals (E) except for yttrium) were washed with chloroform, bathed for ten minutes in caustic lye of soda (20%), rinsed with deionized water and dried.

A 5% by volume solution of bis-[3-(triethoxysilyl)-propyl]tetrasulfide was deposited in an aqueous ethanol solution. The ratio of the bis-silane to water and ethanol was (5)/(5)/(90) volume fractions. The solution was stirred and left at room temperature for at least 48 hours.

Furthermore, a suspension of SiO₂ nanoparticles with an average particle diameter of 15 nanometers was prepared in water, wherein the colloidal solution obtained contained 0.03% by weight of the SiO₂ nanoparticles.

Subsequently a mixture of 5 parts of the colloidal nanoparticle-containing solution and 95 parts of the bis-silane was produced. The concentration of the silicon dioxide particles in the homogenous colloidal silane solution obtained was 15 ppm.

The stent was immersed in this solution for 30 seconds, removed again, rinsed with deionized water and dried for one hour at 100° C.

### EXEMPLARY EMBODIMENT 3

### Serial Coating of a Stent with Two bis-silanes

Stents made of the biocorrodible magnesium alloy WE43 (93% by weight magnesium, 4% by weight yttrium (W) and 3% by weight rare earth metals (E) except for yttrium) were washed with chloroform, bathed in caustic lye of soda (20%) for ten minutes, rinsed with deionized water and dried.

A 5% by volume solution of a 4:1 mixture of bis-[3-(trimethoxysilylpropyl)]amine and vinyltriacetoxysilane was placed in deionized water. The solution was stirred and left at room temperature for at least 48 hours.

Furthermore a 5% by volume solution of bis-[3-(triethoxysilyl)-propyl]tetrasulfide was placed in an aqueous, ethanol solution. The ratio of the bis-silane to water and ethanol was (5)/(5)/(90) volume fractions. The solution was stirred and left at room temperature for at least 48 hours.

Furthermore a suspension of SiO₂ nanoparticles with an average particle diameter of 1 μm in was prepared in water, wherein the colloidal solution obtained contained 0.03% by weight of the SiO₂ nanoparticles.

Subsequently a mixture of 5 parts of the colloidal nanoparticle-containing solution and 95 parts of the respective bis-silane solution was produced. The concentration of the silicon dioxide particles in the homogenous colloidal silane solution obtained was 15 ppm.

The coating was carried out in a serial manner: 15 seconds in the nanoparticle-containing bis-sulfur-silane solution, subsequently 15 seconds in the nanoparticle-containing bis-amino-silane solution and again in the nanoparticle-containing bis-sulfur-silane solution for 15 seconds. The stents were then rinsed with deionized water and dried at 100° C. for twelve hours.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. Method for producing an implant of a biocorrodible metallic material with a nanoparticle-containing silane coating, wherein the method comprises the steps:
(i) Providing a blank for the implant comprising the biocorrodible metallic material, and
(ii) Coating the blank with a homogenous colloidal solution of
A) one or more bis-silanes of the formula (1):
(RO)₃—Si—X—Si(OR)₃ (1)
wherein R stands for C₁-C₁₀ alkyl or C₂-C₁₀ acyl; and
X is a substituted or unsubstituted C₁-C₂₀ alkanediyl group or a substituted or unsubstituted C₁-C₂₀ heteroalkanediyl group with 1 to 5 heteroatoms selected from the group O, N and S; and
B) Silicon dioxide nanoparticles with an average particle diameter in the range of 10 nm to 1 μm in water, ethanol or a mixture of the same, wherein the solution contains 1 to 20% by volume bis-silane and the concentration of the silicon dioxide nanoparticles in the solution is in the range of 1 to 100 ppm.

2. Method according to claim 1, in which the biocorrodible metallic material is a biocorrodible alloy selected from the group comprising magnesium, iron, zinc and tungsten.

3. Method according to claim 2, in which the biocorrodible metallic material is a magnesium alloy.

4. Method according to claim 1, in which R is selected from the group comprising methyl, ethyl, propyl and i-propyl.

5. Method according to claim 4, in which the one or more bis-silanes comprise bis-[3-(triethoxysilyl)-propyl]tetrasulfide and/or bis-[3-(trimethoxysilyl-propyl)]amine.

6. Method according to claim 4, in which the homogenous colloidal solution contains a mixture of bis-[3-(trimethoxysilylpropyl)]amine and vinyltriacetoxysilane.

7. Method according to claim 1, in which step (ii) is repeated n-fold, wherein n=2 to 10 and the homogenous colloidal solution used in each repetition step can be freely selected in terms of its composition.

8. Implant of a biocorrodible metallic material with a nanoparticle-containing silane coating, wherein the coating comprises
A) one or more bis-silanes of the formula (1):
(RO)₃—Si—X—Si(OR)₃ (1)
wherein R stands for C₁-C₁₀ alkyl or C₂-C₁₀ acyl; and
X is a substituted or unsubstituted C₁-C₂₀ alkanediyl group or a substituted or unsubstituted C₁-C₂₀ heteroalkanediyl group with 1 to 5 heteroatoms selected from the group O, N and S; and
B) Silicon dioxide nanoparticles with an average particle diameter in the range of 10 nm to 1 μm.

9. Implant according to claim 8, in which the biocorrodible metallic material is a biocorrodible alloy selected from the group comprising magnesium, iron, zinc and tungsten.

10. Implant according to claim 9, in which the biocorrodible metallic material is a magnesium alloy.

11. Implant according to claim 8, in which the implant is a stent.

12. Implant according to claim 8, in which R is selected from the group comprising methyl, ethyl, propyl and i-propyl.

13. A method for producing an implant of a biocorrodible metallic material with a nanoparticle-containing silane coating, wherein the method comprises the steps:
providing a blank made from a biocorrodible alloy selected from the group comprising magnesium, iron, zinc and tungsten; and
repeatedly coating the blank with a homogenous colloidal solution containing about 1-20% by volume of one or more of bis-[3-(triethoxysilyl)-propyl]tetrasulfide and bis-[3-(trimethoxysilyl-propyl)]amine, and about 1 to 100 ppm silicon dioxide nanoparticles with an average particle diameter in the range of 10 nm to 1 μm, and containing one or more of water and ethanol.
